(19) United States
(12) Patent Application Publication
(10) Pub. No.: US 2008/0153840 A1
(43) Pub. Date: Jun. 26, 2008

(54) REDUCTION OF CARDIOVASCULAR SYMPTOMS

(76) Inventors: Luiz Belardinelli, Palo Alto, CA (US); Lin Wu, Sunnyvale, CA (US); Sridharan Rajamani, Fremont, CA (US); Arvinder Dhalla, Mountain View, CA (US)

Correspondence Address: CV THERAPEUTICS, INC., 3172 PORTER DRIVE, PALO ALTO, CA, 94304, US

(21) Appl. No.: 11/961,860

(22) Filed: Dec. 20, 2007

Related U.S. Application Data
(60) Provisional application No. 60/876,331, filed on Dec. 21, 2006.
(60) Provisional application No. 60/898,019, filed on Jan. 29, 2007.

(51) Int. Cl. A61K 31/496; A61P 9/00

(52) U.S. Cl. 514/252.12

(57) ABSTRACT

The present application discloses methods for treating patients with a dysfunction of the ion channels of the heart suffering from one or more cardiovascular diseases to reduce the proarrhythmic effect of drugs and/or conditions that reduce or inhibit I_{Kr}.

This application claims priority to U.S. Provisional Patent Application Ser. No. 60/876,331, filed Dec. 21, 2006, and to U.S. Provisional Patent Application Ser. No. 60/898,019, filed Jan. 29, 2007, the entireties of both of which are incorporated herein by reference.

### FIELD OF THE INVENTION

This invention relates to methods for treating patients with a dysfunction of the ion channels of the heart suffering from one or more cardiovascular diseases to reduce the proarrhythmic effect of drugs and/or conditions that reduce or inhibit I_{Kr}, comprising administering ranolazine to these patients. In one embodiment, the presenting patient exhibits down-regulated or inhibited I_{Kr}, caused by drugs including, but not limited to, E-4031, clofilium, dofetilide and cisapride. In a further embodiment the presenting patient exhibits down-regulated or inhibited I_{Kr} caused by a dysfunction of the ion channels of the heart such as LQTS.

### DESCRIPTION OF THE ART

U.S. Pat. No. 4,567,264, the specification of which is incorporated herein by reference in its entirety, discloses ranolazine, (±)—N-(2,6-dimethylphenyl)-4-[2-hydroxy-3-(2-methoxyphenoxy)-propyl]-1-piperazineacetamide, and its pharmaceutically acceptable salts, and their use in the treatment of cardiovascular diseases, including arrhythmias, variant and exercise-induced angina, and myocardial infarction. In its dihydrochloride salt form, ranolazine is represented by the formula:

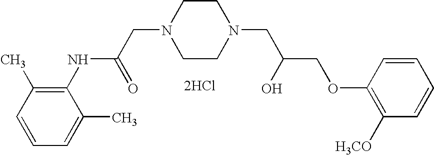

This patent also discloses intravenous (IV) formulations of dihydrochloride ranolazine further comprising propylene glycol, polyethylene glycol 400, Tween 80 and 0.9% saline.

U.S. Pat. No. 5,506,229, which is incorporated herein by reference in its entirety, discloses the use of ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of ranolazine and microcrystalline cellulose coated with release controlling polymers. This patent also discloses IV ranolazine formulations which at the low end comprise 5 mg ranolazine per milliliter of an IV solution containing about 5% by weight dextrose. And at the high end, there is disclosed an IV solution containing 200 mg ranolazine per milliliter of an IV solution containing about 4% by weight dextrose.

The presently preferred route of administration for ranolazine and its pharmaceutically acceptable salts and esters is oral. A typical oral dosage form is a compressed tablet, a hard gelatin capsule filled with a powder mix or granulate, or a soft gelatin capsule (softgel) filled with a solution or suspension. U.S. Pat. No. 5,472,707, the specification of which is incorporated herein by reference in its entirety, discloses a high-dose oral formulation employing supercooled liquid ranolazine as a fill solution for a hard gelatin capsule or softgel.

U.S. Pat. No. 6,503,911, the specification of which is incorporated herein by reference in its entirety, discloses sustained release formulations that overcome the problem of affording a satisfactory plasma level of ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, the specification of which is incorporated herein by reference in its entirety, discloses methods of treating cardiovascular diseases, including arrhythmias variant and exercise-induced angina and myocardial infarction.

U.S. Patent Application Publication Number 2006/0177502, the specification of which is incorporated herein by reference in its entirety, discloses oral sustained release dosage forms in which the ranolazine is present in 35-50%, preferably 40-45% ranolazine. In one embodiment the ranolazine sustained release formulations of the invention include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

### BACKGROUND

Ranolazine is an approved anti-ischemic agent with a proposed mechanism of action to reduce cardiac ischemia by inhibition of the late Na⁺ current (late I_{Na}) and its attendant sequelae of increases of cellular sodium, sodium-calcium exchange, and intracellular calcium loading. Ranolazine has been reported to inhibit I_{Kr} in dog and guinea-pig ventricular myocytes, whereas inhibition of the slow component of the delayed rectifier K⁺ current (I_{Kr}) was minimal (˜17% at 30 μM) or no effect on the inward rectifier K⁺ current (I_{KI}) and transient outward K⁺ current (Iₜₒ). Results of studies with rabbit and guinea-pig isolated hearts and canine ventricular myocytes show that ranolazine causes a concentration-dependent increase in the monophasic APD (MAPD) or QT interval, that is small (˜2-6 msec) at therapeutic concentrations, and is not associated with pro-arrhythmic activity.

There is a condition known as abnormal prolongation of ventricular repolarization, or long QT Syndrome (LQTS), which is reflected by a longer than average interval between the Q wave and the T wave as measured by an EKG. Prolongation of the QT interval renders patients vulnerable to a very fast, abnormal heart rhythm (an “arrhythmia”) known as Torsade de Pointes (TdP). When this kind of arrhythmia occurs, no blood is pumped out from the heart, and the brain quickly becomes deprived of oxygen, causing sudden loss of consciousness (syncope) and potentially leading to sudden death.

LQTS is caused by heritable or acquired dysfunction of the ion channels of the heart. These channels control the flow of potassium ions, sodium ions, and calcium ions, the flows of which in and out of the cells generate the electrical activity of the heart. Patients with LQTS usually have no identifiable underlying structural cardiac disease. LQTS may be inherited, with the propensity to develop a particular variety of ventricular tachycardia under certain circumstances, for example exercise, the administration of certain pharmacological agents, or even during sleep. Alternatively, patients may acquire LQTS, for example by exposure to certain prescription medications. One form of LQTS is LQT2.

The amplitude of late sodium current (I_{Na}) is increased by mutations in the cardiac sodium channel that are found in patients with LQT 3 syndrome. Although normally small, I_{Na} is increased in ventricular muscle cells from failing hearts, from hearts exposed to hypoxia or to metabolites found in ischemic myocardium, and from hearts of animals expressing the mutant sodium channels that are found in patients with LQT 3 syndrome. Patients with LQT 3 and ventricular myocardium in which I_{Na} is present are especially sensitive to the proarrhythmic effect of drugs and conditions that reduce the repolarising current I_{Kr}. However, the contribution of I_{Na} to the increase of duration of the QT interval and to proarrhythmia in the presence of I_{Kr} blockers has not been elucidated.

Recently Wu, et al[J. Pharmacol. & Exper. Thera. 316(2), 718-726 (2006)] described a series of drugs (cisapride, quinidine, zipasidone, moxifloxicin, pentobarbital, and ranolazine) that have different pharmacological activities, but share an effect to inhibit I_{Kr}. Cisapride, a motility enhancer that can be used to treat patients with gastroesophageal reflux disease, and quinidine, a class Iₐ antiarrhythmic agent, have been shown to prolong the QT interval and to trigger TdP. Moxifloxacin has been reported to cause QT prolongation, but its use has been very rarely associated with TdP in humans. Ziprasidone is an antipsychotic agent that has been reported to prolong the QT interval and cause arrhythmias, but the risk of arrhythmic activity associated with its use is likely to be very low. Pentobarbital, a barbitureate, and ranolazine, an antianginal agent, have been shown to prolong the QT interval without causing TdP in cardiac preparations from laboratory animals or in clinical use.

Drug-induced QT prolongation is most commonly associated with drugs that inhibit the rapid component of the delayed rectifier potassium current, I_{Kr}. The use of drugs that prolong the QT interval is considered to increase the risk of TdP in humans. However, the incidence of TdP caused by drugs that prolong the QT interval does not correlate with the extent of QT prolongation by the same drugs.

Ranolazine prolongs QT interval without causing TdP; prolongs MAPD, and inhibits both I_{Kr} and I_{NaL}. Reductions of I_{Kr} and I_{NaL} have opposite actions on action potential duration. In the absence of ATX-II, the inhibition of I_{NaL} by ranolazine predominated and ranolazine increased MAPD₉₀; in the presence of 2 nM ATX-II, the inhibition of I_{NaL} by ranolazine predominated over its action to inhibit I_{Kr}, and ranolazine shortened MAPD. [Wu, et al. [J. Pharmacol. & Exper. Thera. 316(2), 718-726 (2006)].

There is therefore a need for a method for treating patients with a dysfunction of the ion channels of the heart suffering from cardiovascular diseases to reduce the proarrhythmic effect of drugs and/or conditions that reduce or inhibit I_{Kr}.

### SUMMARY OF THE INVENTION

It is an object of this invention to provide methods for treating patients with a dysfunction of the ion channels of the heart suffering from one or more cardiovascular diseases to reduce the proarrhythmic effect of drugs and/or conditions that reduce or inhibit I_{Kr}, comprising the administration of a therapeutically effective amount of ranolazine.

In a first aspect, this invention relates to methods for treating patients with a dysfunction of the ion channels of the heart in which the conditions that reduce or inhibit I_{Kr} include arrhythmias, abnormalities of ventricular repolarization and impaired relaxation of ventricular contraction, comprising the administration of a therapeutically effective amount of ranolazine.

In a second aspect, this invention relates to methods for treating patients with a dysfunction of the ion channels of the heart caused by the presence of a drug or drugs that reduce or inhibit I_{Kr}, comprising the administration of a therapeutically effective amount of ranolazine.

In a third aspect, this invention relates to methods for treating patients with a dysfunction of the ion channels of the heart in which the drugs that reduce or inhibit I_{Kr} include, but are not limited to, E-4031, clofilium, dofetilide, and cisapride.

In a fourth aspect, this invention relates to methods for treating patients with a dysfunction of the ion channels of the heart caused by a physical process occurring in the patient's body, comprising the administration of a therapeutically effective amount of ranolazine.

In a fifth aspect, this invention relates to methods for treating patients with a dysfunction of the ion channels of the heart in which the dysfunction is long QT Syndrome (LQTS).

In a sixth aspect, this invention relates to methods for reversing the APD and arrhymogenesis caused by a pure I_{Kr} blocker, comprising administration of a therapeutically effective amount of ranolazine.

### DETAILED DESCRIPTION OF THE INVENTION

This invention relates to methods for treating coronary patients with a dysfunction of the ion channels of the heart suffering from cardiovascular diseases to reduce the proarrhythmic effect of drugs and/or conditions that reduce or inhibit I_{Kr}, comprising administering ranolazine to these patients. In one embodiment, the presenting patient exhibits down-regulated or inhibited I_{Kr} caused by drugs including, but not limited to, E-4031, clofilium, dofetilide and cisapride. In a further embodiment the presenting patient exhibits down-regulated or inhibited I_{Kr} caused by a dysfunction of the ion channels of the heart such as LQTS.

### DEFINITIONS

In this specification and in the claims that follow, reference will be made to a number of terms that shall be defined to have the following meanings.

“Ranolazine” is the compound (±)-N-(2,6-dimethylphenyl)-4-[2-hydroxy-3-(2-methoxyphenoxy)propyl]-1-piperazine-acetamide, or its enantiomers(R)-(+)-N-(2,6-dimethylphenyl)-4-[2-hydroxy-3-(2-methoxyphenoxy)-propyl]-1-piperazineacetamide, and (S)-(−)-N-(2,6-dimethylphenyl)-4-[2-hydroxy-3-(2-methoxyphenoxy)-propyl]-1-piperazineacetamide, and their pharmaceutically acceptable salts, and mixtures thereof. Unless otherwise stated the ranolazine plasma concentrations used in the specification and examples refer to ranolazine free base. At pH ˜4, in an aqueous solution titrated with hydrogen chloride, ranolazine will be present in large part as its dihydrochloride salt.

“Bradycardia or bradyarrhythmia reducing effective amount” is an amount of ranolazine that treats the bradycardia or bradyarrythmia.

“Physiologically acceptable pH” refers to the pH of an intravenous solution which is compatible for delivery into a human patient. Preferably, physiologically acceptable pH's range from about 4 to about 8.5 and preferably from about 4 to 7. Without being limited by any theory, the use of intravenous solutions having a pH of about 4 to 6 are deemed physiologically acceptable as the large volume of blood in the body effectively buffers these intravenous solutions.

“Cardiovascular diseases” refers to, for example, heart failure, including congestive heart failure, acute heart failure, ischemia, recurrent ischemia, myocardial infarction, NSTEMI, and the like, arrhythmias, angina, including exercise-induced angina, variant angina, stable angina, unstable angina, acute coronary syndrome, NSTEACS, and the like, diabetes, and intermittent claudication. The treatment of such disease states is disclosed in various U.S. patents and patent applications, including U.S. Pat. Nos. 6,503,911 and 6,528,511, U.S. Patent Application Nos. 2003/0220344 and 2004/0063717, the complete disclosures of which are hereby incorporated by reference.

“Topical administration” shall be defined as the delivery of the therapeutic agent to the surface of the wound and adjacent epithelium.

“Parenteral administration” is the systemic delivery of the therapeutic agent via injection to the patient.

“Optional” and “optionally” mean that the subsequently described event or circumstance may or may not occur, and that the description includes instances where the event or circumstance occurs and instances in which it does not. For example, “optional pharmaceutical excipients” indicates that a formulation so described may or may not include pharmaceutical excipients other than those specifically stated to be present, and that the formulation so described includes instances in which the optional excipients are present and instances in which they are not.

“Treating” and “treatment” refer to any treatment of a disease in a patient and include: preventing the disease from occurring in a subject which may be predisposed to the disease but has not yet been diagnosed as having it; inhibiting the disease, i.e., arresting its further development; inhibiting the symptoms of the disease; relieving the disease, i.e., causing regression of the disease, or relieving the symptoms of the disease. The “patient” is a mammal, preferably a human.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, and the age, physical condition, existence of other disease states, and nutritional status of the patient. Additionally, other medication the patient may be receiving will effect the determination of the therapeutically effective amount of the therapeutic agent to administer.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Down-regulated” or “inhibited” refers to the reduction of channel protein expression or inhibition of a current such as the I_{Kr} current. Such down-regulation or inhibition can be caused by presence of a drug (such as an I_{Kr} blocker) or by a physical process occurring in the body (such as LQTS).

E-4031, an I_{Kr} blocker, is an analog of the benzenesulfonamide d-sotalol with greater potency and selectivity than sotalol for inhibition of I_{Kr}. [Tomoto, et al, Cardiovasc. Drugs Ther. 1991 (Suppl. 3): 394; and Lynch, et al. J. Cardiovasc. Pharmacol. 15: 764-775, 1990] E-4031 was designed to be a Class III antiarrhythmic drug based on its action to prolong the duration of the ventricular action potential. However, E-4031 is proarrhythmic and has no current cardiac indication for use.

E-4031 is shown below

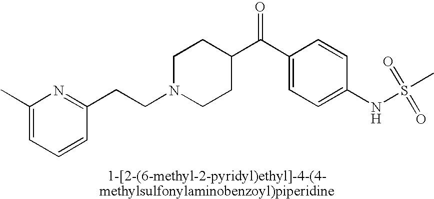

E-4031, also known as {N-(4-[(1-[2-(6-methyl-2-pyridyl)ethyl]-4-piperidylcarbonyl]phenyl)methanesulfonamide dihydrochloride dehydrate}, can be prepared as described in U.S. Pat. No. 4,876,262, the specification of which is incorporated herein by reference.

Ranolazine, which is named N-(2,6-dimethylphenyl)-4-[2-hydroxy-3-(2-methoxyphenoxy)propyl]-1-piperazineacetamide {also known as 1-[3-(2-methoxyphenoxy)-2-hydroxypropyl]-4-[(2,6-dimethylphenyl)-aminocarbonylmethyl]-piperazine}, can be present as a racemic mixture, or an enantiomer thereof, or a mixture of enantiomers thereof, or a pharmaceutically acceptable salt thereof. Ranolazine can be prepared as described in U.S. Pat. No. 4,567,264, the specification of which is incorporated herein by reference.

Tetrodotoxin (TTX, CAS Number [4368-28-9]) is a potent marine neurotoxin, named after the order of fish from which it is most commonly associated, the Tetraodontiformes (tetras-four and odontos-tooth), or the tetraodon pufferfish. Pufferfish from the genus Fugu (F. flavidus, F. poecilonotus, and F. niphobles), Arothron (A. nigropunctatus), Chelonodon (Chelonodon spp.), and Takifugu (Takifugu rubripes) also store TTX and related analogs in their tissues.

TTX is an especially potent neurotoxin, specifically blocking voltage-gated sodium channels on the surface of nerve membranes. The molecule consists of a positively charged guanidinium group (this cationic group is resonance stabilized and gives the name to this class of neurotoxins, q.v., guanidinium toxins) and a pyrimidine ring with additional fused ring systems (these additional ring systems, of which there are five in total, contain hydroxyl groups which must certainly help stabilize the TTX-sodium channel binding complex at the aqueous interface).

The structure of TTX was elucidated simultaneously by an American and two Japanese groups in 1964 (Woodward, R. B., Pure Appl. Chem. 1964, 9, 49-74; Goto T., Kishi Y et al. Tetrahedron 1965, 21, 2059-2088; Tsuda, K., Ikuma, S. et al. Chem. Pharm. Bull, 1964, 12, 1357-1374). The racemic synthesis of TTX was accomplished by Kishi (now at Harvard) and his co-workers in 1972 (Kishi, Y. et al. J. Am. Chem. Soc. 1972, 94).

Clofilium is an example of a potassium channel blocker or inhibitor. This compound, known as 4-chloro-N,N-diethyl-N-heptyl benzenebutanaminium bromide, can be prepared as described in U.S. Pat. No. 4,289,787, the specification of which is incorporated herein by reference. The structure of Clofilium is

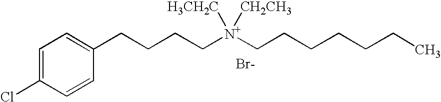

“Immediate release” (“IR”) refers to formulations or dosage units that rapidly dissolve in vitro and are intended to be completely dissolved and absorbed in the stomach or upper gastrointestinal tract. Conventionally, such formulations release at least 90% of the active ingredient within 30 minutes of administration.

“Sustained release” (“SR”) refers to formulations or dosage units used herein that are slowly and continuously dissolved and absorbed in the stomach and gastrointestinal tract over a period of about six hours or more. Preferred sustained release formulations are those exhibiting plasma concentrations of ranolazine suitable for no more than twice daily administration with two or less tablets per dosing as described below.

Methods of this Invention

In one aspect, this invention provides for methods for treating a patient with a dysfunction of the ion channels of the heart suffering from one or more cardiovascular diseases to reduce the proarrhythmic effect of drugs and/or conditions that reduce or inhibit I_{Kr}.

Patients presenting themselves with one or more cardiovascular disease events include, but are not limited to, those who are being treated for one or more of the following: angina including stable angina, unstable angina (UA), exercised-induced angina, variant angina, arrhythmias, intermittent claudication, myocardial infarction including non-STE myocardial infarction (NSTEMI), heart failure including congestive (or chronic) heart failure, acute heart failure, or recurrent ischemia.

Compositions of the Invention

Intravenous Formulation

In one aspect, the invention provides an intravenous (IV) solution comprising a selected concentration of ranolazine. Specifically, the IV solution preferably comprises about 1.5 to about 3.0 mg of ranolazine per milliliter of a pharmaceutically acceptable aqueous solution, more preferably about 1.8 to about 2.2 mg and even more preferably about 2 mg.

Oral Formulation

In one embodiment, a formulation of ranolazine is an oral formulation. In one embodiment, an oral formulation of ranolazine is a tablet. In one embodiment, the tablet of ranolazine is up to 500 mg. In a preferred embodiment, the ranolazine tablet is 375 mg, and/or 500 mg.

The oral formulation of ranolazine is thoroughly discussed in U.S. Pat. No. 6,303,607 and U.S. Publication No. 2003/0220344, which are both incorporated herein by reference in their entireties.

The oral sustained release ranolazine dosage formulations of this invention are administered one, twice, or three times in a 24 hour period in order to maintain a plasma ranolazine level above the threshold therapeutic level and below the maximally tolerated levels, which is preferably a plasma level of about 550 to 7500 ng base/mL in a patient. In a preferred embodiment, the plasma level of ranolazine ranges about 1500-3500 ng base/mL.

In order to achieve the preferred plasma ranolazine level, it is preferred that the oral ranolazine dosage forms described herein are administered once or twice daily. If the dosage forms are administered twice daily, then it is preferred that the oral ranolazine dosage forms are administered at about twelve hour intervals.

In addition to formulating and administering oral sustained release dosage forms of this invention in a manner that controls the plasma ranolazine levels, it is also important to minimize the difference between peak and trough plasma ranolazine levels. The peak plasma ranolazine levels are typically achieved at from about 30 minutes to eight hours or more after initially ingesting the dosage form while trough plasma ranolazine levels are achieved at about the time of ingestion of the next scheduled dosage form. It is preferred that the sustained release dosage forms of this invention are administered in a manner that allows for a peak ranolazine level no more than 8 times greater than the trough ranolazine level, preferably no more than 4 times greater than the trough ranolazine level, preferably no more than 3 times greater than the trough ranolazine level, and most preferably no greater than 2 times trough ranolazine level.

The sustained release ranolazine formulations of this invention provide the therapeutic advantage of minimizing variations in ranolazine plasma concentration while permitting, at most, twice-daily administration. The formulation may be administered alone, or (at least initially) in combination with an immediate release formulation if rapid achievement of a therapeutically effective plasma concentration of ranolazine is desired, or by soluble IV formulations and oral dosage forms.

### Utility Testing and Administration

### General Utility

The compound of the invention is ranolazine which is effective for treating mammals for various disease states, such as for example, heart failure, including congestive heart failure, acute heart failure, ischemia, recurrent ischemia, myocardial infarction, NSTEMI, and the like, arrhythmias, angina, including exercise-induced angina, variant angina, stable angina, unstable angina, acute coronary syndrome, NSTEACS, and the like, diabetes, and intermittent claudication.

### Pharmaceutical Compositions

Ranolazine is usually administered in the form of a pharmaceutical composition. This invention therefore provides pharmaceutical compositions that contain, as the active ingredient, ranolazine, or a pharmaceutically acceptable salt or ester thereof, and one or more pharmaceutically acceptable excipients, carriers, including inert solid diluents and fillers, diluents, including sterile aqueous solution and various organic solvents, solubilizers and adjuvants. Ranolazine may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17^{th} Ed. (1985) and “Modern Pharmaceutics”, Marcel Dekker, Inc. 3^{rd} Ed. (G. S. Banker & C. T. Rhodes, Eds.).

### Administration

Ranolazine may be administered in either single or multiple doses by any of the accepted modes of administration of agents having similar utilities, for example as described in those patents and patent applications incorporated by reference, including buccal, intranasal, intra-arterial injection, intravenously, intraperitoneally, parenterally, intramuscularly, subcutaneously, orally, or as an inhalant.

Oral administration is the preferred route for administration of ranolazine. Administration may be via capsule or enteric coated tablets, or the like. In making the pharmaceutical compositions that include ranolazine, the active ingredient is usually diluted by an excipient and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 50% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Ranolazine is effective over a wide dosage range and is generally administered in a pharmaceutically effective amount. Typically, for oral administration, each dosage unit contains from 1 mg to 2 g of ranolazine, more commonly from 1 to 700 mg, and for parenteral administration, from 1 to 700 mg of ranolazine, more commonly about 2 to 200 mg. It will be understood, however, that the amount of ranolazine actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

In the present example, female rabbit hearts were isolated and paced at 1 Hz and exposed to E-4031 (1-60 nM) in the absence and presence of TTX (0.1-3 μM) and ranolazine (5-30 μM). Monophasic action potentials (MAP) from both epicardium and endocardium, and 12-lead ECG signals were recorded continuously.

E-4031 (1-60 nM), in a concentration-dependent manner, was found to prolong the duration of epicardial MAP (MAPD₉₀) by 75±5 ms from 180±3 to 254±6 ms (n=21, p<0.001), and increase transmural MAPD dispersion by 74±7 ms from 18±4 to 90±10 ms (n=21, p<0.001). Spontaneous or 3-sec pause triggered polymorphic ventricular tachycardias (TdP) occurred in 19 of 21 (90%) hearts studied.

Ranolazine (5-30 μM) was found to concentration-dependently prolong the epicardial MAPD₉₀ by 32±4% (n=7, p<0.01) without increasing transmural dispersion of MAPD₉₀. TTX (0.1-3 μM) had no effects on MAPD₉₀ or transmural dispersion of MAPD₉₀ (n=5, p>0.05).

In the presence of 60 nM E-4031, ranolazine (10 μM) shortened the MAPD₉₀ and decreased the transmural dispersion by 19±5 and 40±9 ms, respectively (n=9, p<0.01). TTX (1 μM) shortened the MAPD and decreased transmural dispersion by 33±8 and 47±11 ms, respectively (n=10, p<0.05). Ranolazine (10 μM) and TTX (1 μM) abolished the spontaneous and pause-triggered episodes of TdP induced by 60 nM E-4031. Ranolazine and TTX caused no or minimal changes in the QRS interval, hence, their effects are unlikely to be due to inhibition of peak I_{Na}.

It was found that inhibition of late I_{Na} can reverse the prolongation of APD and arrhythmogenesis caused by a pure I_{Kr} blocker. The role of late I_{Na} could be significant in hearts when I_{Kr} is inhibited. This finding could explain the observations that sodium channel blockers are effective in reducing arrhythmic activity when I_{Kr} is down-regulated or inhibited.

### EXAMPLE 2

This study was undertaken to investigate the effects of ranolazine (RAN) on clofilium-induced Torsades de Pointes (TdP) in vivo.

Spontaneous TdP was induced with methoxamine (α₁-adrenoceptor agonist) followed by clofilium (Ikr blocker) in anesthetized rabbits as previously described by Carlsson et al (J. Cardiovas. Pharmacol 1990; 16:276-285). RAN was given via intravenous (iv) infusion 10 min prior to clofilium. Arterial pressure (BP), lead II ECG and endocardial Monophasic Action Potential (MAP) were recorded. QT interval was corrected for heart rate by using a formula developed for rabbits: QTc=QT−0.175*(RR−300).

Clofilium prolonged QTc (130±4 to 200±18 ms, n=6, P<0.05), MAPD₉₀ (123±6 to 201±21 ms, n=6, P<0.05) and caused TdP in 6/6 rabbits, which were suppressed by RAN (n=6) in a dose-dependent manner with ED₅₀ values of 10, 15 and 10 μM, respectively. RAN at 25 μM completely prevented the occurrence of TdP (0/6, P<0.005 vs vehicle control) without significant effect on heart rate, PR, QRS and QTc intervals (186±9 to 204±10 bpm, 79±2 to 77±1 ms, 32±1 to 33±1 ms, 148±5 to 165=7 ms, n=5, P>0.05, respectively). Because RAN has been reported to have weak α₁-antagonistic activity we compared the pressor effects of RAN on BP to that of prazosin. While α₁-adrenoceptor antagonist prasozin at 5 μg/kg/min (n=5) markedly shifted phenylephrine dose-response curve to the right by 6 fold, prasozin (2.5-10 μg/kg/min, n=4-6) did not have any effect on clofilium-induced prolongation in QTc and MAPD₉₀ as well as the occurrence of TdP. RAN, on the other hand, completely suppressed TdP but did not cause any shift in phenylephrine dose-response at the highest dose tested.

The data show that RAN antagonizes the ventricular repolarization changes caused by clofilium and is effective in suppressing TdP.

### EXAMPLE 3

Human-ether-a-go-go related gene (HERG) encodes the cardiac rapidly activating delayed rectifier K⁺ current (I_{Kr}). Inhibition of HERG K⁺ current (I_{HERG}) is a mechanism for drug-induced long QT syndrome. This study was undertaken to study the kinetics of ranolazine block of I_{HERG} at 23° C. using voltage-clamp analysis of HERG channels expressed in HEK293 cells. Block of I_{HERG} by ranolazine was reversible and voltage-dependent, but frequency-independent. Block developed rapidly following channel activation, suggesting state-dependence. At 0 mV, the time constants for development of block were 76.6±1.6, 35.8±2.4, and 19.4±1.7 msec in 10, 30, and 100 μM ranolazine (n=4), respectively. The time course of ranolazine-induced I_{HERG} decay was used to estimate the apparent dissociation constant (14.2 μM). Following repolarization, ranolazine-induced block of I_{HERG} reversed rapidly. At −80 and −100 mV, recovery from block followed a monophasic time course with τ values of 204.3±51.5 and 155.0±31.9 msec (n=5), respectively. Intracellular but not extracellular application of a membrane-impermeable (permanently charged) ranolazine analog caused rapid block of I_{HERG}. Ranolazine antagonized E-4031 block of I_{HERG}, suggesting that both compounds compete for a common binding site. Taken together, the unique ultra-rapid kinetics of block (at positive potentials) and unblock (upon hyperpolarization) of I_{HERG} by ranolazine may explain the observations that: (1) ranolazine causes minimal QT interval prolongation with no reverse use dependence, and (2) proarrhythmic events have not been observed during exposures of cardiac myocytes or whole hearts to ranolazine.

### EXAMPLE 4

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### EXAMPLE 5

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### EXAMPLE 6

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules.

### EXAMPLE 7

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets.

### EXAMPLE 8

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### EXAMPLE 9

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### EXAMPLE 10

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### EXAMPLE 11

Suspensions, each containing 50 mg of active ingredient per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### EXAMPLE 12

A subcutaneous formulation may be prepared as follows:

| Ingredient | Quantity | |
|---|---|---|
| Active Ingredient | 5.0 | mg |
| Corn Oil | 1.0 | mL |

### EXAMPLE 13

An injectable preparation is prepared having the following composition:

| Ingredients | Amount | |
|---|---|---|
| Active ingredient | 2.0 | mg/mL |
| Mannitol, USP | 50 | mg/mL |

| | |
|---|---|
| Gluconic acid, USP | q.s. (pH 5-6) |
| water (distilled, sterile) | q.s. to 1.0 mL |
| Nitrogen Gas, NF | q.s. |

### EXAMPLE 14

A topical preparation is prepared having the following composition:

| Ingredients | grams |
|---|---|
| Active ingredient | 0.2-10 |
| Span 60 | 2.0 |
| Tween 60 | 2.0 |
| Mineral oil | 5.0 |
| Petrolatum | 0.10 |
| Methyl paraben | 0.15 |
| Propyl paraben | 0.05 |
| BHA (butylated hydroxy anisole) | 0.01 |
| Water | q.s. to 100 |

All of the above ingredients, except water, are combined and heated to 60° C. with stirring. A sufficient quantity of water at 60° C. is then added with vigorous stirring to emulsify the ingredients, and water then added q.s. 100 g.

### EXAMPLE 15

### Sustained Release Composition

| | Weight | Preferred | Most |
|---|---|---|---|
| Ingredient | Range (%) | Range (%) | Preferred |
| Active ingredient | 50-95 | 70-90 | 75 |
| Microcrystalline cellulose (filler) | 1-35 | 5-15 | 10.6 |
| Methacrylic acid copolymer | 1-35 | 5-12.5 | 10.0 |
| Sodium hydroxide | 0.1-1.0 | 0.2-0.6 | 0.4 |
| Hydroxypropyl methylcellulose | 0.5-5.0 | 1-3 | 2.0 |
| Magnesium stearate | 0.5-5.0 | 1-3 | 2.0 |

The sustained release formulations of this invention are prepared as follows: compound and pH-dependent binder and any optional excipients are intimately mixed (dry-blended). The dry-blended mixture is then granulated in the presence of an aqueous solution of a strong base that is sprayed into the blended powder. The granulate is dried, screened, mixed with optional lubricants (such as talc or magnesium stearate), and compressed into tablets. Preferred aqueous solutions of strong bases are solutions of alkali metal hydroxides, such as sodium or potassium hydroxide, for example sodium hydroxide, in water (optionally containing up to 25% of water-miscible solvents such as lower alcohols).

The resulting tablets may be coated with an optional film-forming agent, for identification, taste-masking purposes and to improve ease of swallowing. The film forming agent will typically be present in an amount ranging from between 2% and 4% of the tablet weight. Suitable film-forming agents are well known to the art and include hydroxypropyl. methylcellulose, cationic methacrylate copolymers (dimethylaminoethyl methacrylate/methyl-butyl methacrylate copolymers—Eudragit® E—Röhm. Pharma), and the like. These film-forming agents may optionally contain colorants, plasticizers, and other supplemental ingredients.

The compressed tablets for example have a hardness sufficient to withstand 8 Kp compression. The tablet size will depend primarily upon the amount of compound in the tablet. The tablets will include from 300 to 1100 mg of compound free base. For example, the tablets will include amounts of compound free base ranging from 400-600 mg, 650-850 mg, and 900-1100 mg.

In order to influence the dissolution rate, the time during which the compound containing powder is wet mixed is controlled. For example the total powder mix time, i.e. the time during which the powder is exposed to sodium hydroxide solution, will range from 1 to 10 minutes and for example from 2 to 5 minutes. Following granulation, the particles are removed from the granulator and placed in a fluid bed dryer for drying at about 60° C.

We claim:

1. A method of reducing cardiovascular disease symptoms in a patient in whom I_{Kr} is reduced or inhibited, comprising the administration of a therapeutically effective amount of ranolazine.

2. The method of claim 1 wherein the cardiac symptom is selected from arrhythmias, abnormalities of ventricular repolarization, and impaired relaxation of ventricular contraction.

3. The method of claim 1 wherein the inhibition of I_{Kr} is caused by the presence of a drug that inhibits I_{Kr}.

4. The method of claim 1 wherein the inhibition of I_{Kr} is caused by a physical process occurring in the patient's body.

5. The method of claim 4 wherein the physical process is congenital LQTS.

6. A method for reversing the APD and arrhymogenesis caused by a pure I_{Kr} blocker, comprising administration of a therapeutically effective amount of ranolazine.
